(19) United States
(12) Patent Application Publication
(10) Pub. No.: US 2010/0178250 A1
(43) Pub. Date: Jul. 15, 2010

(54) Method of Local Delivery of Bioactive and Diagnostic Agents Using Magnetizable Bone Cement

(75) Inventor: Zachary Graham Forbes, Salt Lake City, UT (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Philadelphia Health & Education Corporation, Philadelphia, PA (US)

(21) Appl. No.: 12/438,293

(22) PCT Filed: Aug. 16, 2007
(86) PCT No.: PCT/US07/76126

§ 371 Date: Feb. 20, 2009

Related U.S. Application Data
(60) Provisional application No. 60/823,638, filed on Aug. 25, 2006.

(51) Int. Cl. A61K 49/00; A61K 9/00; A61K 35/12; A61K 38/02; A61K 38/16; A61P 19/00

(52) U.S. Cl. 424/9.3; 424/422; 424/93.7; 514/2; 514/12

(57) ABSTRACT

A method of making a magnetizable implant, the method includes mixing a curable matrix and the at least one of the bioactive agent or the diagnostic agent associated with the magnetizable carrier to form a magnetizable curable matrix; implanting the magnetizable curable matrix in a cavity in a body of a mammal whereby the magnetizable curable matrix takes on a shape of the cavity and forms a molded magnetizable curable matrix; simultaneously curing the molded magnetizable curable matrix and applying the magnetic field and thereby causing the at least one of the bioactive agent or the diagnostic agent associated with a magnetizable carrier to move and arrange within the molded magnetizable curable matrix at or near an interface between the cavity and an outer surface of the molded magnetizable curable bioactive matrix.

### BACKGROUND OF THE INVENTION

1. Field of Invention

This invention relates to magnetically controllable delivery systems and methods of using thereof to attract and deliver bioactive and diagnostic agents associated with (e.g., attached to, or encapsulated within) magnetizable carriers at selected sites in a body of a mammal. More specifically, this invention relates to the use of magnetizable carriers in connection with orthopedic or dental devices.

2. Description of Related Art

Many attempts have been made to develop an implantable matrix which could facilitate bone or cartilage repair and also deliver bioactive agents such as growth factors or antibiotics. Various approaches to replace bone grafts have included conventional bioresorbable polymers, ceramics such as tricalcium phosphate (TCP), natural polymers, such as collagen, proteoglycans, starches, and hyaluronic acid, and modified bone matrix. To date, these efforts have only produced delivery matrices which may provoke negative tissue reactions, cannot be sterilized, and are difficult to use or manufacture.

Bone cement compositions are useful in the bonding or fixing of an implant material, as well as in the strengthening of damaged natural bone. Such compositions are particularly useful in the areas of orthopedics, dentistry and related medical disciplines. The field of orthopedics deals with bone replacement or defects due to fracture, bone tumors, and other diseases of the bone. Treatment may require surgical resection of all, or part, of a bone. In dentistry applications, a defected jawbone may result from extraction of a tooth, cancer or other diseases.

Bone cement is often used with the implant material in order to bond and affix the implant to the remaining living bone. For example, poly(methyl(meth)acrylate) (PMMA) has been widely used with implants in orthopedics.

Although conventional PMMA bone cement has been used in orthopedic surgery for many years, it is far from ideal because 1) it does not encourage bone in-growth, 2) it is a weaker implement than bone cortex, and 3) it has a high exotherm and monomer toxicity. Research, focusing on bioactive bone cements, has been ongoing to modify or replace conventional PMMA bone cement to eliminate or reduce these limitations.

U.S. Pat. No. 6,593,394 to Li et al. discloses bioactive bone cement comprising a powder component including a strontium phosphate; and a liquid component including Bisphenol A diglycidylether dimethacrylate resin.

U.S. Pat. No. 5,336,700 to Murray describes using PMMA cement powder in mixing PMMA dental and orthopedic cements in preparation of implants.

U.S. Pat. No. 6,299,905 to Peterson et al. discloses an implantable matrix for tissue repair which comprises a bio-erodable polymer mixed with a bioactive agent.

Bone cement acts like a grout and not so much like a glue in arthroplasty. Although sticky, it primarily fills the spaces between the prosthesis and the bone preventing motion. It has a Young's modulus between cancellous bone and cortical bone. Thus, bone cement is a load sharing entity in the body not causing bone resorption (see Wu et al., Drug/device combinations for local drug therapies and infection prophylaxis, Biomaterials 27 (2006) 2450-2467)).

Hydroxylapatite can be used as a filler to replace amputated bone or as a coating to promote bone ingrowth into prosthetic implants. Although many other phases exist with similar or even identical chemical makeup, the body responds much differently to them. Coral skeletons can be transformed into hydroxylapatite by high temperatures; their porous structure allows relatively rapid ingrowth at the expense of initial mechanical strength. The high temperature also burns away any organic molecules such as proteins, preventing host vs. graft disease.

Bone infection (osteomyelitis) is a local or generalized infection of bone and bone marrow typically caused by bacteria introduced from trauma, surgery, use of implant, by direct colonization from a proximal infection, or via systemic circulation. Osteomyelitis caused by an implant is clinically difficult to treat. The biofilm mode of pathogen growth on an implant surface protects sessile bacterial colonies against host immune response and antimicrobial therapy through complex environmental factors. Conventional therapy with systemic antibiotics is expensive, prone to complications, and often unsuccessful. Major problems treating osteomyelitis include poor antimicrobial distribution at the site of infection due to limited blood circulation to infected skeletal tissue, and inability to directly address the biofilm pathogen scenario.

High systemic dosage of antibiotics to facilitate sufficient tissue and biofilm penetration is not preferable due to possible serious toxic side effects. Controlled antimicrobial release systems in orthopedic combination devices represent alternatives to conventional systemic treatments, and include antibiotic-eluting bioceramics, drug-impregnated bone cements, and natural and synthetic antimicrobially loaded polymers.

One commonly used infection management method with orthopedic implants utilizes antibiotics loaded into clinically ubiquitous bone cement, polymethylmethacrylate (PMMA), or PMMA beads. These non-biodegradable polymer cements have been employed clinically to prevent or treat osteomyelitis in various forms for nearly four decades (1). Several commercial antibiotic-impregnated bone cements based primarily on PMMA/MMA are now CE-approved, including SIMPLEX P (P. Wu, D. W. Grainger/Biomaterials 27 (2006) 2450-2467) with erythromycin and colistin tobramycin (Stryker, UK) sold in Europe for more than 20 years, and gentamicin-containing PALACOS PMMA cement (refobacin palaces r-Knochenzements, Merck, Austria). A gentamicin-containing PMMA bead, Septopals (E. Merck, Germany), is also commercially available in Europe. In 2003, the first pre-blended bone cement containing an antibiotic (SIMPLEX P with tobramycin developed by Stryker Howmedica Osteonics (Kalamazoo, Mich.) was approved for use in the United States. Later in 2003, Biomet, Inc. (Warsaw, Ind.) announced FDA clearance of their PALACOS GTM antibiotic-loaded bone cement.

The most important growth factors with potential for bone repair and regeneration are morphogenetic proteins (BMP), transforming growth factor beta (TGF-b), insulin-like growth factors (IGF), fibroblast growth factors (FGF), platelet-derived growth factor (PDGF) and vascular endothelial growth factor (VEGF). A detailed description of their biological and clinical roles in development and repair of the skeleton is available (13). Growth factor delivery has been studied using diverse platform technologies and materials, in different bone defects and various animal models.

PMMA can be loaded to deliver a variety of widely used antimicrobials and some other bioactive “agents” including anti-osteoporetic agents, proteins (model protein, albumin) and peptides (e.g., growth factors). Loaded drugs are usually released in a typical bi-phasic fashion: an initial burst release followed by a long, tail of low, and importantly, largely incomplete release that continues for days to months. Small molecule antimicrobial release behavior from PMMA is influenced by relative loading amount, bulk porosity, surface area and surface roughness of the bone cement. Addition of soluble lactose to PMMA produces increased antimicrobial release by percolation-based porous diffusion. All of these observations lead to the conclusion that PMMA bone cement drug release occurs through solvent pore penetration, soluble matrix dissolution, and solubilized drug outward diffusion via networks of continuous, accessible pores within an otherwise largely insoluble, dense, glassy bulk PMMA matrix. In vivo studies have demonstrated that antimicrobial-loaded bone cement can prevent infection from intraoperative challenge within a short time after implantation. Effectiveness in preventing infections is further illustrated in prospective, randomized, and controlled clinical trials comparing antibiotic-loaded bone cement to drug-free bone cement control groups. Tobramycin is an aminoglycoside closely related to gentamicin with a similar spectrum of activity, slightly more effective against Pseudomonas, but less ototoxic and nephrotoxic than gentamicin. Tobramycin's elution characteristics are judged superior to those of gentamicin. A recent clinical study testing the pharmacokinetics and safety profile of tobramycin bone cement demonstrated local tobramycin concentrations more than 200 times higher than systemic levels only 1 h after administration. Systemic drug absorption was minimal with rapid urine excretion.

However, there are drawbacks to use of antimicrobial-loaded bone cement. For example, gentamicin and tobramycin are used most frequently by surgeons for incorporation into bone cement in Europe and United States, respectively. Pharmacokinetic studies indicate that antibiotic release from gentamicin-impregnated PMMA cement or beads is far from satisfactory. Less than 50% of the antibiotic load is released from implants within 4 weeks, and no continuous release was observed thereafter indicating significant bioavailability problems. Recently, 19 of 28 bacterial strains cultured directly from clinically retrieved gentamicin-loaded bone cement were gentamicin resistant, raising concerns for the effectiveness of gentamicin-incorporated implants.

Regardless of the different antimicrobial agents mixed into PMMA liquid resins and its long tradition in orthopedic device fixation, inherent limitations reduce clinical enthusiasm for these combination implants. PMMA is not biodegradable, so with any clinical failure, secondary surgery is necessary to remove the PMMA before new bone can regenerate in the defect. PMMA polymerization exhibits a well-known, prominent exotherm. Both this heat and residual MMA monomer can kill healthy surrounding bone cells and possibly inactivate the antibiotic if PMMA is used in the popular “dough like” form. Other criticisms are the low PMMA bonding strength to the implant surface and known soft tissue encapsulation of PMMA. In cases of loosening and removal, bone substance will also be lost. Biomimetic synthetic hydroxyapatites (HAP) are a more attractive natural candidate as composite materials for bone cement due to their intrinsic non-toxicity, high biocompatibility, and ability to support growth of new bone tissue. HAP attempts to produce the same elementary inorganic chemical solid chemical composition as bone and tooth mineral. Past work investigated release behavior of cephalexin- and norfloxacin-loaded HAP cement in vitro. Drug release patterns of these antibiotic-loaded HAP cements correlated well with the Higuchi model. The 4.8 wt % norfloxacin-loaded cement provided continuous antibiotic release to 250 h with complete release estimated to be 3 weeks. Anionic collagen: HAP composite pastes for antibiotic controlled release have been developed using inorganic salts, Ca(NO₃)₂ (4H₂O) and (NH₄)2PO₄, mixed with anionic collagen at a mass ratio of 20:1 followed by addition of ciprofloxacin. Antibiotic release rate is controlled by the porosity and tortuosity in the composite, permitting drug release throughout the healing process. Other synthetic hydroxyapatite cements such as b-tricalcium phosphate or calcium phosphate bioceramics, either alone or associated with natural or synthetic polymers have also been studied to treat bone infection with some claims to success. These composites provide potential bulk compositional versatility for magnetic carrier based antibiotic-releasing formulations.

The ability to apply forces on magnetic particles with external magnetic fields has been harnessed in various biomedical applications including prosthetics (Herr, H. J. of Rehab. Res. and Devel. 2002 39(3):11-12), targeted drug delivery (Goodwin, S. J. of Magnetism and Magnetic Materials 1999 194:209-217) and antiangiogenesis strategies (Liu et al. J. of Magnetism and Magnetic Materials 2001 225:209-217; Sheng et al. J. of Magnetism and Magnetic Materials 1999 194:167-175). U.S. Pat. No. 4,247,406 describes an intravascularly-administrable, magnetically-localizable biodegradable carrier comprising microspheres formed from an amino acid polymer matrix containing magnetic particles embedded within the matrix for targeted delivery of chemotherapeutic agents to cancer patients. Microspheres with magnetic particles, which are suggested to enhance binding of a carrier to the receptors of capillary endothelial cells when under the influence of a suitable magnetic field, are also described in U.S. Pat. No. 5,129,877.

U.S. Pat. Nos. 6,375,606; 6,315,709; 6,296,604; and 6,364,823 describe methods and compositions for treating vascular defects, and in particular aneurysms with a mixture of biocompatible polymer material, biocompatible solvent, adhesive and preferably magnetic particles to control delivery of the mixture. In these methods, a magnetic coil or ferrofluid is delivered via catheter into the aneurysm. This magnetic device is shaped, delivered, steered and held in place using external magnetic fields and/or gradients. This magnetic device attracts the mixture to the vascular defect wherein it forms an embolus in the defect thereby occluding the defect.

A model for inducing highly localized phase transformations at defined locations in the vascular system by applying 1) external uniform magnetic fields to an injected superparamagnetic colloidal fluid for the purpose of magnetization and 2) using embedded particles to create high magnetic field gradients was described by inventors (Forbes et al. Abstract and Poster Presentation at the 6th Annual New Jersey Symposium on Biomaterials, Oct. 17-18, 2002, Somerset, N.J.). This work describes the use of uniform magnetic fields in combination with large magnetic particles (greater than 2 micron in diameter) to form chains along the direction of applied field and in turn use this to embolize micro-vessels (50-100 microns in diameter). The use of these magnetizable implants in drug delivery was also described previously by authors Z. Forbes, B. B. Yellen, G. Friedman, and K. Barbee (IEEE Trans. Magn. 39(5): 3372-3377 (2003)).

Chen (U.S. Pat. No. 5,921,244) discloses inserting a magnet (an electromagnet or a permanent magnet) or a plurality of magnets into an opening in a body to attract magnetic fluid/particles. The plurality of magnets is described to be disposed along the longitudinal axis of the magnetic probe.

Gordon (U.S. Patent Publication No. US 2002/0133225) describes a device comprising an implant having a magnetic field and a medical agent carried by a magnetically sensitive carrier. The carrier is introduced into the blood flow of the organism upstream from the target tissue, and the carrier and medical agent migrate via the blood flow to the target tissue. Gordon discloses an implant comprising a magnetized material (e.g., a ferromagnetic or a superparamagnetic material). Examples describe making a stent from ferromagnetic materials and magnetized by using an external magnet or made from a magnetized material.

Despite the foregoing developments, there is still a need in the art for improved methods of delivery of therapeutic agents utilizing magnetic forces.

All references cited herein are incorporated herein by reference in their entireties.

### BRIEF SUMMARY OF THE INVENTION

Accordingly, a first aspect of the invention includes a method of making a magnetizable implant, the method comprising:

(a) providing a curable matrix;

(b) providing at least one of a bioactive agent or a diagnostic agent associated with a magnetizable carrier;

(c) mixing the curable matrix and the at least one of the bioactive agent or the diagnostic agent associated with the magnetizable carrier to form a magnetizable curable matrix;

(d) implanting the magnetizable curable matrix in a cavity in a body of a mammal whereby the magnetizable curable matrix takes on a shape of the cavity and forms a molded magnetizable curable matrix;

(e) providing to the molded magnetizable curable matrix an external source of a magnetic field capable of magnetizing the magnetizable carrier; and

(f) simultaneously curing the molded magnetizable curable matrix and applying the magnetic field and thereby causing the at least one of the bioactive agent or the diagnostic agent associated with a magnetizable carrier to move and arrange within the molded magnetizable curable matrix at or near an interface between the cavity and an outer surface of the molded magnetizable curable bioactive matrix and thereby making the bioactive magnetizable implant having a layer of the at least one of the bioactive agent or the diagnostic agent substantially in a shape of the cavity disposed at the interface of the cavity and the outer surface of the molded magnetizable curable matrix.

A second aspect of the invention comprises a bioactive magnetizable implant made by the method described above, wherein the bioactive magnetizable implant comprises the molded magnetizable curable matrix having the bioactive agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity.

A third aspect of the invention comprises a diagnostic magnetizable implant made by the method described above, wherein the diagnostic magnetizable implant comprises the molded magnetizable curable matrix having the diagnostic agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity and wherein the diagnostic agent is other than magnetizable carrier.

A fourth aspect of the invention includes a method of delivering at least one of a bioactive agent or a diagnostic agent to a cavity in a body of a mammal, the method comprising:

(a) providing a curable matrix;

(b) providing the at least one of the bioactive agent or the diagnostic agent associated with a magnetizable carrier;

(c) mixing the curable matrix and the at least one of the bioactive agent or the diagnostic agent to form a magnetizable curable matrix;

(d) implanting the magnetizable curable matrix in a cavity in a body whereby the magnetizable curable matrix takes on a shape of the cavity and forms a molded magnetizable curable matrix;

(e) providing to the molded magnetizable curable matrix an external source of a magnetic field capable of magnetizing the magnetizable carrier; and

(f) simultaneously curing the molded magnetizable curable matrix and applying the magnetic field and thereby causing the at least one of the bioactive agent or the diagnostic agent associated with a magnetizable carrier to move and arrange within the molded magnetizable curable matrix at or near an interface between the cavity and an outer surface of the molded magnetizable curable matrix; and

(g) forming the magnetizable implant, wherein the bioactive magnetizable implant comprises the molded magnetizable curable bioactive matrix having the bioactive agent associated with the magnetizable carrier, wherein the magnetizable carrier is arranged within the molded magnetizable curable bioactive matrix at or near an interface between the cavity and the outer surface of the molded magnetizable curable bioactive matrix as a layer substantially in a shape of the cavity and thereby delivering at least one of the bio active agent or the diagnostic agent to the cavity in the body of the mammal.

### BRIEF DESCRIPTION OF THE DRAWING

FIG. 1 is a cross section of a leg in total hip arthroplasty, which is a schematic representation of the preferred embodiment of the invention utilizing a bioactive magnetizable implant comprising a magnetizable carrier in a shape of particles.

### DETAILED DESCRIPTION OF PREFERRED EMBODIMENTS OF THE INVENTION

The invention was driven by a desire to provide a reliable method of delivering bioactive agents and/or diagnostic agents to a specific location in a body. The inventor has discovered that desired bioactive agents and/or diagnostic agents can be efficiently and reliably delivered using bone or dental cement to improve integration of orthopedic or dental implants into the body. This invention addresses a need to prevention infection around the implant or to expedite bone growth around the implant using, for example, mitogenic or morphogenic drugs. Advantageously, this invention facilitates a larger, more efficient dose and allows for the delivery of subsequent doses in cases of implant infection, loosening, or other complications. Advantageously, the site of the implantation can be imaged due to the presence of the imaging agents in the matrix (bone or dental cement).

A first aspect of the invention includes a method of making a magnetizable implant, the method comprising (a) providing a curable matrix; (b) providing at least one of a bioactive agent or diagnostic agent associated with a magnetizable carrier; (c) mixing the curable matrix and the at least one of a bioactive agent or diagnostic agent associated with the magnetizable carrier to form a magnetizable curable matrix; (d) implanting the magnetizable curable matrix in a cavity in a body of a mammal whereby the magnetizable curable matrix takes on a shape of the cavity and forms a molded magnetizable curable matrix; (e) providing to the molded magnetizable curable matrix an external source of a magnetic field capable of magnetizing the magnetizable carrier; and (f) simultaneously applying the magnetic field and curing the molded magnetizable curable matrix and thereby causing the at least one of a bioactive agent or diagnostic agent associated with a magnetizable carrier to move and arrange within the molded magnetizable curable matrix at or near an interface between the cavity and an outer surface of the molded magnetizable curable matrix and thereby making the magnetizable implant having a layer of the at least one of a bioactive agent or diagnostic agent substantially in a shape of the cavity disposed at the interface of the cavity and the outer surface of the molded magnetizable curable matrix.

In certain embodiments, commercially produced bone cement made of poly(methyl methacrylate) or hydroxyapatite, or otherwise polymerizable bone cement material is magnetized using nanometer, micrometer, millimeter, or centimeter sized particles of magnetizable materials. The magnetizable materials may be of a soft-magnetic, paramagnetic, ferromagnetic, or superparagmagnetic nature. The magnetizable particles may be encapsulated within a biological or pharmaceutical polymer, such as, for example, dextran, or poly(lactic glycolic) acid (PLGA), or other biodegradable material. Encapsulated particles or clumps of magnetic material can be bound with or encapsulate bioactive agents (e.g., antibiotics, antiseptics, radioactive agents, biological cells, anti-neoplastics, anti-inflammatories, mitogenic drugs, morphogenic drugs, or other therapeutic agents) or diagnostic agents (e.g., contrast agents, diagnostic radiopharmaceuticals, etc.).

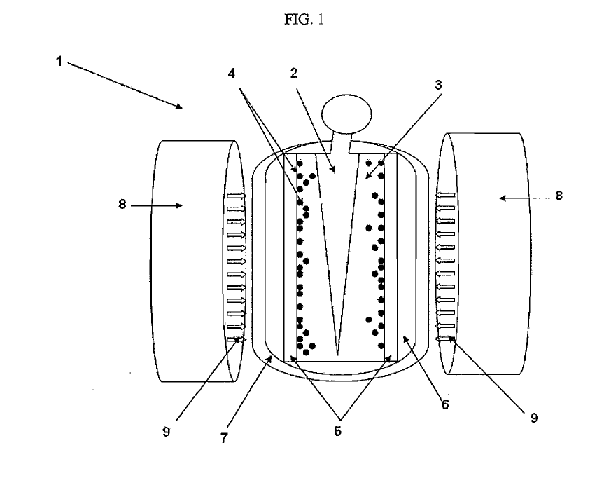

The addition of the magnetizable or magnetic materials can occur by adding a dehydrated dispersion to the polymer powder, or by suspending the magnetizable or magnetic material within the monomer fluid. The mixing of the two agents and the beginning of the polymerization will allow for uniform mixing of the magnetic material within the matrix of the curing bone cement. After the bone cement is rapidly inserted into a patient, magnetic field is applied over the surrounding tissue. This magnetic field may be a sheet of magnetic rubber, a continuous segmentation of permanent magnetic material (with rare earth metals such as neodymium, samarium cobalt, or otherwise), a discontinuous segmentation of permanent magnetic material (with rare earth metals such as neodymium, samarium cobalt, or otherwise), an orientation of electro magnets around the tissue, or other source of external magnetic field as shown in FIG. 1.

In certain embodiments where a bioactive agent is added to the matrix, the magnetizable implant is a bioactive magnetizable implant. In certain embodiments, where a diagnostic agent is added to the matrix, the magnetizable implant is a diagnostic magnetizable implant. In certain embodiments, both a bioactive agent and a diagnostic agent are added. In certain embodiments, an additional bioactive agent and/or a diagnostic which are not associated with magnetizable carriers are added.

The magnetizable implant of the invention made from bone cement and bioactivated magnetizable carrier plays a role of a bioactive/diagnostic agent carrier at implantation wherein the location of the bioactive/diagnostic agent associated with the magnetizable carrier is determined by the application of magnetic force and boundaries created by the implant during the curing process. An assembly (1) in accordance with the invention is shown in FIG. 1 as a cross section of a leg in total hip arthroplasty. After insertion of the hip implant (2) and uncured magnetizable curable bioactive matrix (bone cement mixed with magnetizable carriers (4) and the bioactive agent associated with the carriers) (3), an external source of a magnetic field (9) in a shape of a sleeve (8) is fastened over the leg (not shown). The sleeve (9) contains a strong, rare earth metal magnetizable or magnetic material (e.g., magnetic coils). During the curing process, the magnetic fields (9) draw the magnetizable carriers (4) to the interface of bone (5) with bone cement (3), allowing migration of the magnetizable carriers to the interface and migration of the bioactive agent associated with the carriers to and beyond the interface (e.g., to the bone 5, muscle/fat (6), and skin (7)).

The externally applied magnetic field uniformly orients these magnetic materials along the interface of the bone and the bone cement. After curing is completed, the magnetic material becomes fixed in place at or near an interface between the cavity and an outer surface of the molded magnetizable curable bioactive matrix (the implant). Consequently, the bioactive/diagnostic magnetizable implant has a layer of the bioactive/diagnostic agent substantially in a shape of the cavity, which is disposed at the interface of the cavity and the outer surface of the molded magnetizable curable matrix. Such disposition of the desired bioactive/diagnostic agent is advantageous because it delivers the desired agent at the location prone to infections or other events associated with the healing process.

Subsequent doses of magnetically-bound bioactive/diagnostic agents can be provided by parenteral administration of loaded magnetizable particles (i.e., magnetizable particles associated with bioactive/diagnostic agents. Such loaded magnetizable particles will be captured near the bioactive magnetizable implant, by the gradients of the magnetic material within the cement, when an external magnetic field is applied.

### DEFINITIONS

By the terms “magnetizable carrier” and “magnetizable particle”, as used herein, it is meant a carrier or a particle made from materials that conduct magnetic flux strongly. The term “a magnetizable particle” is used interchangeably with the term “magnetic carrier” and the term “magnetic particle” throughout this disclosure. Examples of magnetizable carriers or particles useful in the present invention include, but are not limited to, cobalt, iron, iron oxides, nickel, manganese, and rare earth magnetic materials (e.g., samarium and neodymium) and various soft magnetic alloys (e.g., Ni—Co). In one embodiment, the magnetizable carrier or particle is magnetized only in the presence of externally applied magnetic fields. Examples of these types of magnetizable materials include, but are not limited to, superparamagnets and soft ferromagnets. In other embodiment, magnetizable materials known as ferromagnets, which can be permanently magnetized, are used.

The magnetizable carrier or particle of the invention can be prepared by methods known in the art in various shapes and sizes (see, for example Hyeon T., Chemical Synthesis of Magnetic Nanoparticles. The Royal Society of Chemistry 2003, Chem. Commun., 2003, 927-934). In certain embodiments, iron oxide nanocrystals were obtained by precipitation of mixed iron chlorides in the presence of a base in aqueous medium (see Khalafalla S E. Magnetic fluids, Chemtech 1975, September: 540-547).

Magnetizable carriers can be in a shape of particles, crystals, spheres, rods, wires, blocks, pellets, or other dispersions. Magnetizable materials are added to the curable matrix of the invention (e.g., bone cement) to make the matrix magnetizable.

In certain embodiments of the method, the magnetizable carrier is a magnetizable particle with a diameter from about 10 nm to about 1000 nm. Preferably, the magnetizable particle has a diameter from 10 nm to 500 nm.

Exemplary magnetizable particles Spherotech (Spherotech, Ill.) have 20% γ-Fe2O3 magnetite by weight a nominal diameter of 350 nm with approximately 10% variance in size. These particles have a carboxylate per nm² of surface area, which can be used as a linker for bioactive or diagnostic agents with corresponding reactive functional groups.

In certain embodiments of the method, the magnetizable particle comprises a cell such that the magnetizable particle is loaded within a cell and the bioactive agent is associated with the cell, the magnetizable particle or both. Magnetizable nanoparticles can be delivered into cells by endocytosis.

Those skilled in the art would be able to select material for making the magnetizable carrier or particle such that it would be magnetized in the presence of an external magnetic field as those materials are known or are being developed (e.g., metals, metal alloys and rear earth elements). In certain embodiments, the magnetizable carrier or particle is made from at least one of materials selected from the group consisting of cobalt, nickel, iron, manganese, samarium and neodymium.

In certain embodiments, the magnetizable carrier or particle contains a support made from a metal, a rare earth element, a ceramic, a polymer or a combination thereof. In certain embodiments, the magnetizable carrier or particle contains a coating on the support, wherein the coating is made from a metal, a rare earth element, a ceramic, a polymer or a combination thereof. A coating is defined below and is preferably made from a magnetizable material. For example, if the support is not made from magnetizable material, the coating must be made from a magnetizable material.

The magnetizable carrier can be made by coating any suitable support with a magnetizable coating by methods known in the art such as, for example, electrodeposition or electrospraying.

The term “coating”, as used herein, includes coatings that completely cover a surface, or a portion thereof (e.g., continuous coatings, including those that form films on the surface), as well as coatings that may only partially cover a surface, such as those coatings that after drying leave gaps in coverage on a surface (e.g., discontinuous coatings). The later category of coatings may include, but is not limited to a network of covered and uncovered portions. Coatings can be flat or raised above the surface or embossed on the surface (e.g., a ridge) or it can be in a shape of dots or other shapes creating a pattern. A combination of various coatings can also be used.

Coating can be made from a magnetizable material (e.g., stainless steel, soft magnetic alloys) and a non-magnetizable material (a polymer). Selecting the appropriate combination of coating and support materials, it is desirable that the magnetizable carrier or particle has a set of segments on its surface that will enable the creation of a localized magnetic gradient. For example, if the support is made from a magnetizable compound, material(s) of the segment can have a higher or a lower degree of magnetization or they can be made from non-magnetizable materials. On the other hand, if the support or a surface of the magnetizable object is made from a non-magnetizable compound, material(s) of the segment must be made from a magnetizable compound.

It should be understood that the benefits of the bioactive magnetizable implant of the invention must not come at the cost of increased risk in other areas, such as chemical tolerance of a magnetic coating or final compositions of polymer and magnetite crystals. It is preferred to utilize FDA approved magnetic or magnetizable particle composites, as well as soft magnetic coatings and magnetic alloys in order to explore the range of manufacturing capabilities that maintain the fundamental essence of the technology such as utilization of the bioactive magnetizable implant and controllable local delivery of magnetizable particles loaded with a desired substance (e.g., a drug and/or a cell or a diagnostic agent) to the bioactive magnetizable implant. While both soft magnetic coatings and varied alloy composition appear to possess functionality for adapting implants to this magnetic drug delivery system, it is possible that their chemical effects and responses to MRI will differ. As biocompatibility is important in clinical testing, this system provides desired flexibility in the design which makes it much more attractive to the industry.

Regarding MRI, a technology is being developed which uses magnetic material to enhance MRI safety and quality (Biophan, Mass.). This opens the possibility of achieving a balance between such enhancements and the point of magnetization of an implant that would create safety issues relative to movement or torquing of the implant. The current invention provides enough flexibility in the design that the options of patient receiving an MRI would not be compromised. One skilled in the art using the guidance provided in this disclosure would be able to design a bioactive magnetizable implant system that would not preclude safe and effective MRI procedures for patients receiving the implants in accordance with the invention. Similar concerns can be addressed for other types of treatment or diagnostic methods wherein magnetic interference may be a problem.

### Curable Matrix/Bone Cement

The term “a curable matrix”, as used herein, includes a polymeric material capable of being cured or polymerized by, for example, initiators, heat or radiation.

The term “bone cement” as used herein, includes any suitable bone cement useful in orthopedic or dental applications. Exemplary bone cements include those described by U.S. Pat. No. 6,593,394 to Li et al and U.S. Pat. No. 5,336,700 to Murray, which are incorporated herein in their entireties.

In orthopedics, an acrylate (e.g., poly(methylmethacrylate) (PMMA)) based bone cement is used to affix implants and to remodel lost bone. It is supplied as a powder with liquid methyl methacrylate (MMA). When mixed together, PMMA and MMA yield a dough-like cement that gradually hardens in the body. Surgeons can judge the curing of the PMMA bone cement by the smell of MMA in the patient's breath. Although PMMA is biologically compatible, MMA is considered to be an irritant and a possible carcinogen. PMMA has also been linked to cardiopulmonary events in the operating room due to hypotension (1).

The powder used in making the cement typically includes fine particles of poly(methylmethacrylate) (PMMA), poly(methylmethacrylate co-styrene) polymer, and benzoyl peroxide. Barium sulfate is optionally added to provide X-ray opacity and may constitute approximately 10 percent by weight of the powder. The benzoyl peroxide acts as a chemical initiator and may constitute approximately 2 percent by weight of the cement powder. The cement powder is primarily very small rounded particles of PMMA and PMMA styrene co-polymer. Orthopedic cement powder also includes exceedingly fine particles of PMMA and PMMA styrene co-polymer. Dental cement powder typically does not include the exceedingly fine particles.

The methylmethacrylate (MMA) monomer liquid mixed with the cement powder typically includes dimethyl-p-toluidine and hydro-quinone. The dimethyl-p-toluidine is a cold-curing agent which may constitute approximately 2.6 percent by weight of the liquid. The hydroquinone is a stabilizer usually added in very small amounts.

PMMA cement powder is mixed directly with the MMA monomer liquid in a ratio of approximately 40 grams of powder to 20 ml. of liquid. Mixed cement is should be used prior solidification, i.e., during approximately 10 minutes after the start of mixing. The short useful life of the cement requires rapid mixing of the cement and delivering the cement to the application site.

Both the liquid and powder components may contain the conventional additives in this field. Thus, for example, the powder component may contain minor amounts of an X-ray contrast material, polymerization initiators and the like. The liquid component may contain crosslinking agents and minor amounts of polymerization inhibitors, activators, color agents, and the like.

In this invention, magnetizable materials (e.g., crystals, spheres, rods, wires, blocks, pellets, or other dispersions) associated with bioactive or diagnostic agents are added to bone cement to make the bone cement magnetizable either to a liquid or a powder component or to both. Magnetizable materials are preferably added prior to mixing the components.

It is also contemplated for certain embodiments to use bioactive and/or diagnostic agents which are not associated with magnetizable materials. Such agents can be added at any stages of preparing the curable matrix, added prior, contemporarily or after addition of the agents associated with the magnetizable materials.

In certain embodiments, bone cement is prepared as described by U.S. Pat. No. 4,910,259 to Kindt-Larsen et al., which is incorporated herein in its entirety. In those embodiments, the liquid component contain at least three distinct (meth)acrylate monomers. The three groups are listed below along with certain of the preferred materials: (1) C₁-C₂ Alkyl methacrylates (e.g., methylmethacrylate and ethylmethacrylate), (2) straight or branched long chain (meth)acrylates having a molecular weight of at least 168 and preferably 6 to 18 carbon atoms in the straight or branched chain substituents (e.g., n-hexylmethacrylate, n-heptylmethacrylate, ethylhexylmethacrylate, n-decylmethacrylate, isodecylmethacrylate, lauric methacrylate, stearic methacrylate, polyethyleneglycolmethacrylate, polypropyleneglycolmethacrylate, and ethyltriglycolmethacrylate), and (3) Cyclic (meth)acrylates having a molecular weight of at least 168 and preferably 6 to 18 carbon atoms in the cyclic substituents (e.g., cyclohexymethacrylate, benzylmethacrylate, iso-bornylmethacrylate, adamantylmethacrylate, dicyclopentenyloxyethylmethacrylate, dicyclopentenylmethacrylate, dicyclopentenylacrylate, 3,3,5-trimethylcyclohexylmethacrylate, and 4-tert-butylcyclohexylmethacrylate).

As noted above, the liquid component or phase may contain crosslinking agents and minor amounts of additives such as polymerization inhibitors, activators, and the like. The polymerization inhibitors may be hydroquinone, hydroquinonemonomethylether, ascorbic acid, mixtures thereof, and the like in amounts ranging from about 10 to 500 ppm, preferably 20 to 100 ppm w/w. The activator is employed in amounts ranging from 0.2 to 3.0% w/w, preferably 0.4 to 1.0%, and may be N,N-dimethyl-p-toluidine, N,N-hydroxypropyl-p-toluidine, N,N-dimethyl-p-aminophen ethanol, N,N,-diethyl-p-aminophenyl acetic acid, and the like. It has been found helpful to use a combination of N,N-dimethyl-p-toluidine and N,N-hydroxypropyl-p-toluidine. Most preferably, the latter compound is used in greater proportions, e.g. 2 parts by weight for each part of N,N-dimethyl-p-toluidine. Useful crosslinking agents include ethyleneglycol dimethacrylate, 1,4-butanediol dimethacrylate, 1,3-butanediol dimethacrylate, triethyleneglycol dimethacrylate, tetraethyleneglycol dimethacrylate, polyethyleneglycol-400 dimethacrylate, neopentylglycol dimethacrylate, bisphenol A dimethacrylate, ethoxylated Bisphenol A dimethacrylate, trimethylolpropane trimethacrylate, and tripropyleneglycol acrylate.

The powder component or phase comprises a (meth)acrylate polymer, copolymer or a mixture of both. Illustrative materials include polyethylmethacrylate, polyisopropylmethacrylate, poly-sec-butylmethacrylate, poly-iso-butylmethacrylate, polycyclohexylmethacrylate, poly(butylmethacrylate-co-methylmethacrylate), poly(ethylmethacrylate-co-methylmethacrylate), poly(styrene-co-butylacrylate), and poly(ethylacrylate-co-methylmethacrylate).

The polymer powder may be utilized in finely divided form such as, for example, 20 to 250 microns. Admixed with the solid material may be X-ray contrast, polymerization initiator, antibiotics, antiseptic additives, and the like. Conventional X-ray contrast additives such as barium sulphate, zirconium dioxide, zinc oxide, and the like are used in amounts ranging from 5 to 15% w/w. Typical polymerization initiators can be used in amounts ranging from about 0.5 to 3.0% w/w. Examples of such initiators are benzoyl peroxide, lauroyl peroxide, methyl ethyl peroxide, diisopropyl peroxy carbonate. It will be understood that neither the use of most of the aforementioned additives nor the amounts thereof constitute essential features of the present invention. Moreover, the bone cement may also containing filler materials such as carbon fibers, glass fibers, silica, alumina, boron fibers, and the like.

The weight ratio of the liquid monomer component and the polymer powder component will range from about 1 to about 2.5, 1 to 1.5, and preferably from 1 to 2.

As is well known in the art the final bone cement composition is obtained by mixing the liquid monomeric component with the free-flowing, polymeric powder component. The materials are admixed and dispensed in the conventional manner using known equipment.

Bone cement acts like a grout and not so much like a glue in arthroplasty. Although sticky, it primarily fills the spaces between the prosthesis and the bone preventing motion. It has a Young's modulus between cancellous bone and cortical bone. Thus, bone cement is a load sharing entity in the body without causing bone resorption (1).

Another example of a suitable matrix material is hydroxylapatite which can be used as a filler to replace amputated bone or as a coating to promote bone in-growth into prosthetic implants. Hydroxylapatite, also frequently called hydroxyapatite, is a naturally occurring form of calcium apatite with the formula Ca₅(PO₄)₃(OH), but is usually written Ca₁₀(PO₄)₆(OH)₂ to denote that the crystal unit cell comprises two molecules. The OH⁻ ion in the apatite group can be replaced by fluoride, chloride or carbonate. It crystallizes in the hexagonal crystal system. It has a specific gravity of 3.08 and is 5 on the Mohs hardness scale. Hydroxylapatite is the main mineral component of dental enamel, dentin, and bone.

Although many other phases exist with similar or even identical chemical makeup, the body responds much differently to them. Coral skeletons can be transformed into hydroxylapatite by high temperatures; their porous structure allows relatively rapid ingrowth at the expense of initial mechanical strength. The high temperature also burns away any organic molecules such as proteins, preventing host vs. graft disease.

The term “a dental cement” or “a dental composite” as used herein, includes a composition which, after being cured, is stable and bonds well to hard tissues such as tooth enamel and dentin and to prostheses such as inlays, onlays, crowns, cores, posts and bridges that are formed of metals, porcelains, ceramics and composite resins, and which is therefore useful in restoring decayed or injured teeth and in bonding prostheses. An exemplary composition is described in U.S. Pat. No. 6,984,673 to Kawashima et al., which is incorporated herein in its entirety.

In certain embodiments bone cement can be used for dental applications and vise versa as a person skilled in the art would appreciate.

### Bioactive Agent

The term “a bioactive agent”, as used herein, means any organic or inorganic agent that is biologically active, e.g., produces some biological affect in a subject.

In certain embodiments of the composition, the bioactive agent is a member selected from the group consisting of a nucleic acid, a protein, a peptide, an oligonucleotide, an antibody, an antigen, a viral vector, a bioactive polypeptide, a polynucleotide coding for the bioactive polypeptide, a cell regulatory small molecule, a gene therapy agent, a gene transfection vector, a receptor, a cell, a drug, a drug delivering agent, an antimicrobial agent, an antibiotic, an antimitotic, an antisecretory agent, an anti-cancer chemotherapeutic agent, steroidal and non-steroidal anti-inflammatories, a hormone, a proteoglycan, a glycosaminoglycan, a free radical scavenger, an iron chelator, a radiotherapeutic agent, and an antioxidant.

Preferred bioactive agents include antibiotics, antiseptics, anti-inflammatories, anti-neoplastics, mitogenic and morphogenic agents, cells (stem cells and differentiated cells), growth factors, growth hormones, morphogenic proteins and morphogenic protein stimulatory factors.

Exemplary antibiotics may be active against gram-negative bacteria, active against both gram-positive and gram negative bacteria. Preferably, the antibiotic is active against gram-positive bacteria. Exemplary antibiotics include but are not limited to minocyclins, tigecycline tetracycline, glycylcycline, vancomycin and its analogs, rifampicin and its family members, methcillin and its analogs, gentamycin and its analogs, tobramycin and its analogs and combinations of several antibiotics.

Exemplary anti-inflammatory agents include steroidal agents (e.g., substances related to cortisone, like methylprednisolone acetate) and non-steroidal agents (e.g., acetylsalicyclic acid, ibuprofen, acetaminophen, indomethacin, celecoxib, and rofecoxib).

The term “bone morphogenetic protein (BMP)” refers to a protein belonging to the BMP family of the TGF-beta superfamily of proteins (BMP family) based on DNA and amino acid sequence homology. A protein belongs to the BMP family according to this invention when it has at least 50% amino acid sequence identity with at least one known. BMP family member within the conserved C-terminal cysteine-rich domain which characterizes the BMP protein family. Members of the BMP family may have less than 50% DNA or amino acid sequence identity overall.

The term “morphogenic protein” refers to a protein having morphogenic activity (see below). Preferably, a morphogenic protein of this invention comprises at least one polypeptide belonging to the BMP protein family. Morphogenic proteins may be capable of inducing progenitor cells to proliferate and/or to initiate differentiation pathways that lead to cartilage, bone, tendon, ligament, neural or other types of tissue formation depending on local environmental cues, and thus morphogenic proteins may behave differently in different surroundings. For example, an osteogenic protein may induce bone tissue at one treatment site and neural tissue at a different treatment site.

Exemplary morphogenic proteins are described in U.S. Pat. No. 7,026,292 to Lee et al, which is incorporated herein in its entirety. Morphogenic proteins are capable of stimulating a progenitor cell to undergo cell division and differentiation, and that inductive activity may be enhanced in the presence of a MPSF.

Many mammalian morphogenic proteins have been described. Some fall within a class of products called “homeodomain proteins”, named for their homology to the drosophila homeobox genes involved in phenotypic expression and identity of body segments during embryogenesis. Other morphogenic proteins are classified as peptide growth factors, which have effects on cell proliferation, cell differentiation, or both.

The term “osteogenic protein (OP)” refers to a morphogenic protein that is capable of inducing a progenitor cell to form cartilage and/or bone. The bone may be intramembranous bone or endochondral bone. Most osteogenic proteins are members of the BMP protein family and are thus also BMPs. However, the converse may not be true. BMPs (identified by sequence homology) must have demonstrable osteogenic activity in a functional bioassay to be osteogenic proteins according to this invention.

The term “morphogenic protein stimulatory factor (MPSF)” refers to a factor that is capable of stimulating the ability of a morphogenic protein to induce tissue formation from a progenitor cell. The MPSF may have a direct or indirect effect on enhancing morphogenic protein inducing activity. For example, the MPSF may increase the bioactivity of another MPSF. Agents that increase MPSF bioactivity include, for example, those that increase the synthesis, half-life, reactivity with other biomolecules such as binding proteins and receptors, or the bioavailability of the MPSF.

The terms “morphogenic activity”, “inducing activity” and “tissue inductive activity” alternatively refer to the ability of an agent to stimulate a target cell to undergo one or more cell divisions (proliferation) that may optionally lead to cell differentiation. Such target cells are referred to generically herein as progenitor cells. Cell proliferation is typically characterized by changes in cell cycle regulation and may be detected by a number of means which include measuring DNA synthetic or cellular growth rates. Early stages of cell differentiation are typically characterized by changes in gene expression patterns relative to those of the progenitor cell, which may be indicative of a commitment towards a particular cell fate or cell type. Later stages of cell differentiation may be characterized by changes in gene expression patterns, cell physiology and morphology. Any reproducible change in gene expression, cell physiology or morphology may be used to assess the initiation and extent of cell differentiation induced by a morphogenic protein.

Exemplary growth factor families useful in this invention include TGF-beta (transforming growth factor-beta), BMP (bone morphogenic protein), neurotrophins (NGF, BDNF, and NT3), fibroblast growth factor (FGF), myostatin (GDF-8), and platelet-derived growth factor (PDGF).

Exemplary stem cells include cord blood stem cells and somatic stem cells. Exemplary differentiated cells include osteocytes, chondrocytes, and adipocytes and endothelial cells.

Preferred are bone and cartilage forming cells such as, for example, osteoblasts and osteocytes.

### Diagnostic Agent

The term “diagnostic agent” as used herein includes an agent usable in diagnostics by methods known in the art, such as, for example, imaging methods (e.g., MRI, X-ray, etc.).

Exemplary diagnostic agents include a paramagnetic metal ion (e.g., of atomic number 21 to 29, 42, 44 and 57 to 71, especially 24 to 29 and 62 to 69), a heavy metal ion (e.g., of atomic number 37 or more preferably 50 or more) or an ion of a radioactive metal isotope. Preferred paramagnetic metal ions are Eu, Ho, Gd, Dy, Mn, Cr and Fe, and particularly preferred paramagnetic ions are Gd(III), Mn(II) and Dy(III). Preferred heavy metal ions are Hf, La, Yb, Dy and Gd. Preferred radioactive isotopes are useful for scintigraphy, SPECT or PET imaging. For use in PET imaging, one of the various positron emitting metal ions, such as ⁵¹Mn, ⁵²Fe, ⁶⁰Cu, ⁶⁸Ga, ⁷²As, ^{94m}Tc, or ¹¹⁰In is preferred. Preferred isotopes for labeling by halogenation include ¹⁸F, ¹²⁴I, ¹²⁵I, ¹³¹I, ¹²³I, ⁷⁷Br, and ⁷⁶Br. Preferred radioactive metal isotopes for scintigraphy include ⁶⁴Cu, ⁶⁷Ga, ⁶⁸Ga, ⁸⁷Y, ^{99m}Tc, and ¹¹¹In. It should be understood that in embodiments where only diagnostic agent is added, the diagnostic agent is made of a material other than the magnetizable carrier.

Bioactive or Diagnostic Agent Associated with Magnetizable Carrier/Particle

The bioactive or diagnostic agent to be used in the method of the invention is encapsulated in, attached to, or dispersed in a magnetizable carrier/particle. For example, the therapeutic agent may be encapsulated in magnetic particles including, but not limited to, microspheres and nanospheres or magnetic liposomes. Alternatively, the bioactive or diagnostic agent may be dispersed in a ferrofluid or in a colloidal fluid. In embodiments wherein the magnetic carrier involves magnetic particles and/or liposomes to be used outside of the curable matrix, it is preferred that the particles and/or liposomes be less than 10 micrometers in size to prevent clogging of any small arterioles.

Selection of a bioactive or diagnostic agent to be encapsulated within the magnetic carrier such as magnetic particles or magnetic liposomes or dispersed in a magnetic carrier such as ferrofluid and used with the devices of the present invention is dependent upon the use of the device and/or the condition being treated and the site of implantation of the magnetizable device.

In embodiments concerning with attachment of bioactive or diagnostic agent, a covalent bonding is preferred. Magnetizable particles can be treated to contain suitable reactive groups such as for example, hydroxy, carboxy or amino groups with would be reactive with suitable functional groups of bioactive agents. A person skilled in the art would be able to select suitable materials based on known methods. Exemplary magnetizable particles Spherotech (Spherotech, Ill.) have 20% γ-Fe2O3 magnetite by weight a nominal diameter of 350 nm with approximately 10% variance in size. These particles have a carboxylate per nm² of surface area, which can be used as a linker for bioactive or diagnostic agents with corresponding reactive functional groups.

The magnetizable curable bioactive matrix is formed by mixing the curable matrix with the bioactive agent associated with the magnetizable carrier. Similarly, a magnetizable curable matrix or is formed by mixing the curable matrix and the diagnostic agent associated with the magnetizable carrier. Magnetizable materials associated with either bioactive agents or diagnostic agents or both can are added to the curable matrix of the invention (e.g., bone cement) to make it magnetizable at various stages of making the bone cement.

The term “an external source of a magnetic field capable of magnetizing the magnetizable carrier” as used herein, includes, for example, an electromagnet.

In a preferred embodiment, the external source of the magnetic field is in a shape of an article capable of being worn on a part of a body within the closest distance from the cavity with the bioactive magnetizable implant. Non-limiting examples of such article include a band which can be placed around a knee if the implant is located in the knee or around a hip if the implant is in the hip.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “therapeutically effective amount” as used herein means an amount sufficient to impart the desired therapeutic effect to the subject in need thereof.

### Bioactive Magnetizable Implant/Diagnostic Magnetizable Implant

A second aspect of the invention comprises a bioactive magnetizable implant made by the method described above, wherein the bioactive magnetizable implant comprises the molded magnetizable curable bioactive matrix having the bioactive agent associated with the magnetizable carrier, wherein the magnetizable carrier is arranged within the molded magnetizable curable bioactive matrix at or near an interface between the cavity and the outer surface of the molded magnetizable curable bioactive matrix as a layer substantially in a shape of the cavity.

A third aspect of the invention comprises a diagnostic magnetizable implant made by the method described above, wherein the diagnostic magnetizable implant comprises the molded magnetizable curable matrix having the diagnostic agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity and wherein the diagnostic agent is other than magnetizable carrier.

It should be understood that the amounts of magnetizable carriers and bioactive agent can be varied depending on applications and that arranging of the magnetizable carriers along the interface depends on the strength of the magnetic field applied and the time between the insertion of the matrix and total cure of the matrix. It should also be understood that in some embodiments, not all magnetizable carriers will reach the interface and can still be found within the matrix.

### Bone Cement Applications

The magnetizable curable matrix of the invention may be injected into the vertebral body for treatment of spinal fractures, injected into long bone or flat bone fractures to augment the fracture repair or to stabilize the fractured fragments, or injected into intact osteoporotic bones to improve bone strength. It is also useful in the augmentation of a bone-screw or bone-implant interface. Additionally, it is useful as bone filler in areas of the skeleton where bone may be deficient. Examples of situations where such deficiencies may exist include post-trauma with segmental bone loss, post-bone tumor surgery where bone has been excised, and after total joint arthroplasty. It is further useful as a cement to hold and fix artificial joint components in patients undergoing joint arthroplasty, as a strut to stabilize the anterior column of the spine after excision surgery, and as a bone graft substitute in spinal fusions.

### Method of Delivering a Bioactive/Diagnostic Agent

Another aspect of the invention includes a method of delivering a bioactive agent and/or a diagnostic agent to a cavity in a body of a mammal, the method includes the following steps: (a) providing a curable matrix; (b) providing at least one of a bioactive agent or a diagnostic agent associated with a magnetizable carrier; (c) mixing the curable matrix and at least one of a bioactive agent or a diagnostic agent to form a magnetizable curable matrix; (d) implanting the magnetizable curable matrix in a cavity in a body whereby the magnetizable curable matrix takes on a shape of the cavity and forms a molded magnetizable curable matrix; (e) providing to the molded magnetizable curable matrix an external source of a magnetic field capable of magnetizing the magnetizable carrier; and (f) simultaneously curing the molded magnetizable curable matrix and applying the magnetic field and thereby causing at least one of the bioactive agent or the diagnostic agent associated with a magnetizable carrier to move and arrange within the molded magnetizable curable matrix at or near an interface between the cavity and an outer surface of the molded magnetizable curable matrix; and (g) forming the magnetizable implant, wherein the magnetizable implant comprises the molded magnetizable curable matrix having at least one of the bioactive agent or the diagnostic agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity.

In certain embodiments, the curable matrix is a bone cement or a dental composite.

In certain embodiments, the magnetizable carrier is at least one of cobalt, iron, iron oxides, nickel, manganese, rare earth magnetic materials and soft magnetic alloys.

In certain embodiments, the external source of the magnetic field is in a shape of an article capable of being worn on a part of a body in a proximity the cavity with the bioactive magnetizable implant. In one variant, the article is at least one of a band which can be placed around a knee if the implant is located in the knee or around a hip if the implant is in the hip.

In certain embodiments, the bioactive agent is at least one of an antibiotic, an antiseptic, an anti-inflammatory, anti-neoplastics, mitogenic and morphogenic agents, cells, growth factors, growth hormones, morphogenic proteins and morphogenic protein stimulatory factors.

In certain embodiments, an additional bioactive agent or a diagnostic which are not associated with magnetizable carriers are added.

In certain embodiments, the method further comprises administering a magnetizable particle capable of being directed to the bioactive magnetizable implant by at least one of the magnetic field created by the external source or a magnetic field created by an internal source which is the bioactive magnetizable implant. In one variant, the magnetizable particle is injected in a vein or an artery. In one variant, the external source of the magnetic field is in a shape of an article capable of being worn on a part of a body in proximity of the cavity with the bioactive magnetizable implant.

### Interactions of Magnetizable Implant and Magnetizable Particles

This invention also relates to orthopedic and dental application of the two-source method for magnetic drug delivery as described in U.S. Patent Application Publication No. US2006-0041182A1 by Forbes et al. incorporated herein in its entirety. The uses of the magnetic drug delivery system are presented for bone cements, and for use of bone cements in conjunction with orthopedic and dental implants other than those made from bone or dental cements, such as, for example, knee, hip, elbow, shoulder, bone pins, bone screws, bone plates and dentures.

In one aspect of the invention, orthopedic or dental implants are manufactured with soft magnetizable (e.g., magnetic or paramagnetic) surface features by, for example, sputtering, electro, or gas mediated deposition. These features may be continuous or patterned as described in U.S. Patent Application Publication No. US2006-0041182A1 by Forbes et al. In addition, magnetizable features can be varied by adjusting the alloy used to compose the implant. For instance, by cold-working steel to allow chromium-carbide precipitates in the resulting material.

After the magnetizable implant of the invention is placed in the body, it can be targeted with magnetic nano- or micro-carriers of bioactive or diagnostic agents, which can be administered parenterally to a subject. With the aid of an externally applied magnetic field to saturate the magnetic moment of the implant as well as the magnetic moment of the injected magnetizable carriers associated with bioactive agents or diagnostic agents, such carriers will be attracted to the magnetizable implant. These carriers may be cells, magnetic cores with therapeutic agents chemically attached to its surface, or a magnetic dispersion within a polymer matrix of a biodegradable material. These carriers may deliver diagnostic agents (e.g., radioactive materials, an imaging agent), and bioactive agents (e.g., antibiotics, antiseptics, mitogenic or morphogenic agents, anti-inflammatories, anti-neoplastics, cells and radiotherapeutic agents).

The invention will be illustrated in more detail with reference to the following Examples, but it should be understood that the present invention is not deemed to be limited thereto.

### Example 1

A preferred embodiment of the invention is shown in FIG. 1. A cross section of a leg in total hip arthroplasty is used to demonstrate the invention. After insertion of the magnetic drug carrier loading bone cement and hip implant, a magnetic sleeve is fastened over the leg containing strong, rare earth metal magnetic material (represented here as magnetic coils). During the curing process, the magnetic fields draw the magnetizable particles to the bone/bone cement interface, allowing delivery of the bioactive agent associated with the particles drug on the surface of the carriers.

Application of magnetic field during curing of bone cement can be done by an externally mounted electromagnet, permanent magnetic materials oriented around the region, or a sleeve/surface placed around the leg/arm/knee or other portion of the body containing the magnetized bone cement. This sleeve can contain magnetic rubber, rare earth metal permanent magnetic material (neodymium, samarium cobalt, or other) capable of producing magnetic fields strong enough to draw the particles to the bone/bone cement interface.

Magnetic field draws magnetic material in bone cement to interface of bone and bone cement.

Pharmaceutical, biological, or radioactive agents are drawn to tissue from the insides of the magnetic material within the bone cement or the surfaces of magnetic material, to elicit therapeutic response.

A bioactive/diagnostic agent is released either by burst release from the surface of the implanted magnetic materials, or continuous release.

In some instances, the use of an externally applied magnetic field may cause the formation of long channels of magnetic particles, uniformly dispersed around the circumference and length of the bone cement/bone interface. This may allow a high, initial burst release of therapeutic agent, with steady release following as particles are drawn out of their pores. In these instances, the use of nano or micro scale magnetic material in the bone cement is preferable in order not to disturb the mechanical integrity of the bone cement and its primary function.

### Example 2

In one embodiment, the magnetic material remains significantly dispersed within the bone cement, allowing future magnetic targeting of magnetic carrier-bound therapeutic agents, using the two source method previously described in U.S. Patent Application Publication No. US2006-0041182A1 by Forbes et al. This form of magnetic material may be encapsulated in a non-biodegradable vehicle such as polystyrene, gold, glass, or other material that will allow it to remain in tact within the bone cement. In this case, the bone cement may be visible by magnetic resonance imaging for diagnosis of complications around the implanted magnetic bone cement.

### Example 3

In another embodiment, the magnetic material is mostly drawn out of the bone cement to be removed from the body, allowing no future magnetic targeting capability.

### Example 4

In another embodiment, the magnetic material bound with drug that resides in the bone cement is also accompanied by unbound drug dispersed within the bone cement.

### Example 5

In another embodiment, the magnetizable material is encapsulated in ultrasound sensitive contrast agents (such as a gas-filled polymer bubble) along with bioactive agents where these contrast agents can be tailored to be sensitive to different frequencies and magnitudes of ultrasound. The cement may be loaded with a variety of magnetized bubbles of varied copolymer and drug content, to allow controlled release of different drugs depending on the ultrasound application.

Exemplary ultrasound sensitive contrast agents useful in this invention are described in U.S. Pat. No. 7,078,015 to Unger. Encapsulation of magnetizable materials along with bioactive agents can be done by using known methods and guidance provided above for polymeric materials.

This would allow, for instance, for physicians to treat complications as the situation demands. In the case of a total hip replacement, a surgeon implants the ultrasound-degradable magnetic particle loaded bone cement, inserts the implant and magnetizes the area to draw the particles out. If and when a physician believed there is infection or inflammation, ultrasound could be used to release antibiotics or antiseptics from dispersion of particles by tailoring the parameters of the applied ultrasound. If and when a physician believed there is aseptic loosening of the implant, the ultrasound could be used to release another dispersion within the cement, containing bioactive agents such as, for example, mitogenic agents, morphogenic agents, or growth hormone, to promote bone growth around the cement. This invention would allow on the spot future treatments by non-invasive means.

### Example 6

Flexural strength of Stryker Simplex PMMA infused with 100 μm magnetic silica particles (Micromod) was tested following ASTM D 790-03 standard procedures. Control samples and 1% magnetic silica particle samples were injection molded into 79.8×10×3.2 mm beams. Samples containing magnetic silica particles were had the appropriate amount of particles mixed into the polymer powder prior to adding the activator and injection into the mold. Samples were left to polymerize for one hour before removing them from the mold.

The mechanical tests were performed on a MTS Mini Bionix Test System according to Procedure A of ASTM D 790-03 with a strain rate of 0.01 mm/mm/min and a span width of 51 mm. Five tests each of the control and magnetic silica particle samples were completed and analyzed to find the modulus of elasticity and stress at fracture. These results can be found in Table 1.

TABLE 1
Modulus of Elasticity and Stress at Fracture data for Control, 0.5% and 1%
microparticle concentration specimens.

| | | | | 0.5% Magnetic | | | 1% Magnetic | | |
|---|---|---|---|---|---|---|---|---|---|
| | Control | | | Microparticles | | | Mircoparticles | | |
| | Modulus of | Strain at | Stress at | Modulus of | Strain at | Stress at | Modulus of | Strain at | Stress at |
| | Elasticity | Fracture | Fracture | Elasticity | Fracture | Fracture | Elasticity | Fracture | Fracture |
| Sample | (MPa) | (mm/mm) | (MPa) | (MPa) | (mm/mm) | (MPa) | (MPa) | (mm/mm) | (MPa) |
| Sample 1 | 2150.20 | 0.0314 | 51.52 | 2161.50 | 0.0337 | 53.47 | 1881.30 | 0.0309 | 43.86 |
| Sample 2 | 2012.70 | 0.0220 | 35.55 | 2025.70 | 0.0247 | 42.79 | 1827.00 | 0.0334 | 47.93 |
| Sample 3 | 2147.20 | 0.0317 | 50.80 | 2025.30 | 0.0354 | 51.19 | 1950.40 | 0.0406 | 47.70 |
| Sample 4 | 2061.40 | 0.0339 | 50.87 | 1925.30 | 0.0328 | 46.56 | 1852.00 | 0.0308 | 43.58 |
| Sample 5 | 2023.70 | 0.0292 | 48.32 | 2132.20 | 0.0274 | 47.50 | 2002.90 | 0.0288 | 44.11 |
| Sample 6 | 2095.60 | 0.0255 | 44.27 | 2035.90 | 0.0312 | 49.93 | 2009.30 | 0.0307 | 46.71 |
| Sample 7 | 2004.00 | 0.0329 | 49.08 | 2016.60 | 0.0250 | 41.89 | 2091.50 | 0.0310 | 50.19 |
| Sample 8 | 2303.70 | 0.0270 | 50.26 | 1953.20 | 0.0293 | 43.96 | 1978.40 | 0.0307 | 46.07 |
| Sample 9 | 2074.80 | 0.0248 | 43.31 | 2003.60 | 0.0304 | 45.76 | 1910.80 | 0.0354 | 46.78 |
| Average | 2097.03 | 0.0287 | 47.11 | 2031.03 | 0.0300 | 47.01 | 1944.84 | 0.0325 | 46.33 |
| Standard | 88.80 | 0.0039 | 4.93 | 71.13 | 0.0036 | 3.70 | 85.07 | 0.0036 | 2.06 |
| Deviation | | | | | | | | | |

An increase in the average modulus of elasticity and stress at fracture for the 1% magnetic microparticle samples has been observed.

### Example 7

Bacterial Cultures

Staphlococcus aureus bacterial cultures were performed in the microbiology department of Hahnemann University Hospital. On each plate 150 μL of control beads, 150 μL, of antibiotic beads and a 10 mg tobramycin disc were placed in order to see any bactericidal activity. It was observed that there are clear areas present around the antibiotic beads and the tobramycin disc indicating lack of bacterial growth.

### Example 8

### Mathematical Model

MatLab was used to model a magnetic particle traveling through an increasingly viscous fluid. The magnetic field was modeled after that of a 2 cm cube of neodymium. The model was a simple 2-D model, assuming a line of neodymium magnets on the medial-lateral sides of the knee. The initial randomized placement of the magnetic particles and their final location after 400 seconds was observed.

While the invention has been described in detail and with reference to specific examples thereof, it will be apparent to one skilled in the art that various changes and modifications can be made therein without departing from the spirit and scope thereof.

What is claimed is:

1. A method of making a magnetizable implant, the method comprising:
(a) providing a curable matrix;
(b) providing at least one of a bioactive agent or a diagnostic agent associated with a magnetizable carver;
(c) mixing the curable matrix and the at least one of the bioactive agent or the diagnostic agent associated with the magnetizable carrier to form a magnetizable curable matrix;
(d) implanting the magnetizable curable matrix in a cavity in a body of a mammal whereby the magnetizable curable matrix takes on a shape of the cavity and forms a molded magnetizable curable matrix;
(e) providing to the molded magnetizable curable matrix an external source of a magnetic field capable of magnetizing the magnetizable carrier; and
(f) simultaneously curing the molded magnetizable curable matrix and applying the magnetic field and thereby causing the at least one of the bioactive agent or the diagnostic agent associated with a magnetizable carrier to move and arrange within the molded magnetizable curable matrix at or near an interface between the cavity and an outer surface of the molded magnetizable curable bioactive matrix and thereby making the bioactive magnetizable implant having a layer of the at least one of the bioactive agent or the diagnostic agent substantially in a shape of the cavity disposed at the interface of the cavity and the outer surface of the molded magnetizable curable matrix.

2. The method of claim 1, wherein the curable matrix is a bone cement.

3. The method of claim 1, wherein the curable matrix is a dental composite.

4. The method of claim 1, wherein the magnetizable carrier is at least one of cobalt, iron, iron oxides, nickel, rare earth magnetic materials or a soft magnetic alloy.

5. The method of claim 1, wherein the external source of the magnetic field is in a shape of an article capable of being worn on a part of a body in a proximity of the cavity with the bioactive magnetizable implant.

6. The method of claim 1, wherein the article is at least one of a band which can be placed around a knee if the implant is located in the knee or around a hip if the implant is in the hip.

7. The method of claim 1, wherein the bioactive agent is added and the magnetizable implant is a bioactive magnetizable implant.

8. The method of claim 7, wherein the bioactive agent is at least one of an antibiotic, an antiseptic, an anti-inflammatory, anti-neoplastics, mitogenic and morphogenic agents, cells, growth factors, growth hormones, morphogenic proteins, and morphogenic protein stimulatory factors.

9. The method of claim 1, wherein the bioactive agent and the diagnostic agent are added.

10. The method of claim 1, wherein the diagnostic agent other than magnetizable carrier is added and the magnetizable implant is a diagnostic magnetizable implant.

11. The method of claim 1, wherein an additional bioactive agent or a diagnostic which are not associated with magnetizable carriers are added.

12. A bioactive magnetizable implant made by the method of claim 1, wherein the bioactive magnetizable implant comprises the molded magnetizable curable matrix having the bioactive agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity.

13. A diagnostic magnetizable implant made by the method of claim 1, wherein the diagnostic magnetizable implant comprises the molded magnetizable curable matrix having the diagnostic agent associated with the magnetizable carrier, wherein the magnetizable carrier is arranged within the molded magnetizable curable matrix at or near an interface between the cavity and the outer surface of the molded magnetizable curable matrix as a layer substantially in a shape of the cavity and wherein the diagnostic agent is other than magnetizable carrier.

14. A method of delivering at least one of a bioactive agent or a diagnostic agent to a cavity in a body of a mammal, the method comprising:
(a) providing a curable matrix;
(b) providing the at least one of the bioactive agent or the diagnostic agent associated with a magnetizable carrier;
(c) mixing the curable matrix and the at least one of the bioactive agent or the diagnostic agent to form a magnetizable curable matrix;
(d) implanting the magnetizable curable matrix in a cavity in a body whereby the magnetizable curable matrix takes on a shape of the cavity and forms a molded magnetizable curable matrix;
(e) providing to the molded magnetizable curable matrix an external source of a magnetic field capable of magnetizing the magnetizable carrier; and
(f) simultaneously curing the molded magnetizable curable matrix and applying the magnetic field and thereby causing the at least one of the bioactive agent or the diagnostic agent associated with a magnetizable carrier to move and arrange within the molded magnetizable curable matrix at or near an interface between the cavity and an outer surface of the molded magnetizable curable matrix; and
(g) forming the magnetizable implant, wherein the bioactive magnetizable implant comprises the molded magnetizable curable bioactive matrix having the bioactive agent associated with the magnetizable carrier, wherein the magnetizable carrier is arranged within the molded magnetizable curable bioactive matrix at or near an interface between the cavity and the outer surface of the molded magnetizable curable bioactive matrix as a layer substantially in a shape of the cavity and thereby delivering at least one of the bioactive agent or the diagnostic agent to the cavity in the body of the mammal.

15. The method of claim 14, wherein the curable matrix is a bone cement.

16. The method of claim 14, wherein the curable matrix is a dental composite.

17. The method of claim 14, wherein the magnetizable carrier is at least one of cobalt, iron, iron oxides, nickel, manganese, rare earth magnetic materials and soft magnetic alloys.

18. The method of claim 14, wherein the external source of the magnetic field is in a shape of an article capable of being worn on a part of a body in a proximity the cavity with the bioactive magnetizable implant.

19. The method of claim 14, wherein the article is at least one of a band which can be placed around a knee if the implant is located in the knee or around a hip if the implant is in the hip.

20. The method of claim 14, wherein the bioactive agent is added.

21. The method of claim 20, wherein the bioactive agent is at least one of an antibiotic, an antiseptic, an anti-inflammatory, anti-neoplastics, mitogenic and morphogenic agents, cells, growth factors, growth hormones, morphogenic proteins and morphogenic protein stimulatory factors.

22. The method of claim 14, wherein the diagnostic agent is added, provided that the diagnostic agent is other than the magnetizable carrier.

23. The method of claim 14, wherein the bioactive agent and the diagnostic agent are added.

24. The method of claim 14, wherein an additional bioactive agent or a diagnostic agent which are not associated with magnetizable carriers are added.

25. The method of claim 14, further comprising administering a magnetizable particle capable of being directed to the bioactive magnetizable implant by at least one of the magnetic field created by the external source or a magnetic field created by an internal source which is the magnetizable implant.

26. The method of claim 25, wherein the magnetizable particle is injected in a vein or an artery.

27. The method of claim 25, wherein the external source of the magnetic field is in a shape of an article capable of being worn on a part of a body in a proximity of the cavity with the magnetizable implant.
